(19) United States
(12) Patent Application Publication
(10) Pub. No.: US 2022/0023461 A1
(43) Pub. Date: Jan. 27, 2022

(54) STERILIZATION DEVICE WITH DOSE FUNCTION

(71) Applicant: INDUSTRIAL TECHNOLOGY RESEARCH INSTITUTE, CHUTUNG (TW)

(72) Inventors: Meng-Hsuan LEE, Huatan Township (TW); Shou-Nan LI, Caotun Township (TW); I-Ling NIEN, Zhubei City (TW); Hui-Ya SHIH, Nantou City (TW); Wen-An XIE, New Taipei City (TW); Chia-Yen KUO, Taipei City (TW); Jui-Hsiang CHENG, Hsinchu City (TW); Chuan-Lin CHANG, Taichung City (TW); Jian-Wei CHENG, Taichung City (TW)

(73) Assignee: INDUSTRIAL TECHNOLOGY RESEARCH INSTITUTE, CHUTUNG (TW)

(21) Appl. No.: 17/090,156

(22) Filed: Nov. 5, 2020

(51) Int. Cl. A61L 2/10; A61L 2/20

(52) U.S. Cl. CPC A61L 2/10; A61L 2/202; A61L 2202/15; A61L 2202/121; A61L 2202/11; A61L 2202/122

(57) ABSTRACT

A sterilization device with dose function is provided. The sterilization device includes a sterilization container, an ultraviolet sterilization module, an ozone sensing module, a sterilization time counter, a dose controller and an ozone removal module. The ultraviolet sterilization module is disposed in the sterilization container to sterilize an article by emitting an ultraviolet light. The ozone sensing module is used to sense an ozone concentration of the sterilization container. The sterilization time counter is used to calculate a sterilization time. The dose controller is used to receive a sensing signal of the ozone concentration and a counting signal of the sterilization time to obtain an ozone dose. The ozone removal module is disposed in the sterilization container to remove or purify the ozone and reduce the ozone concentration in the sterilization container.

This application claims the benefit of Taiwan application Serial No. 109124649, filed Jul. 21, 2020, the disclosure of which is incorporated by reference herein in its entirety.

### TECHNICAL FIELD

The disclosure relates in general to a sterilization device, and more particularly to a sterilization device with dose function.

### BACKGROUND

Due to the rapid growth in population, the Asian Pacific coastal regions have the largest population density in the world. However, the high population density also increases the chances of spreading the epidemics, such as the bird flu, the severe acute respiratory syndrome (SARS), and the corona virus disease 2019 (COVID-19) whose outbreaks over recent years have caused a great social and economic damage to the humanity. Thus, over recent years, personal hygiene awareness arises, home hygiene habits are advocated, and people are paying more attention to public health issues. All these phenomena serve to avoid the cluster transmission of viruses and suppress the spread of pandemic diseases.

Wearing face mask and washing hands frequently is one of the most effective measures to block the infiltration of germs and viruses. However, if the medical masks (before the expiry date) are disposed after use without going through a sterilization process, the germs and viruses will reproduce dramatically. Furthermore, the practice of throwing the masks away immediately after use not only wastes resources but further adds severe burden to waste disposal.

To avoid secondary pollution and at the same time make the medical supplies reusable before the expiry date, it is essential to provide a sterilization device capable of combining the sterilization process and the purification process to reduce the sterilization time and enhance the sterilization and purification effect. Since the ozone concentration in the sterilization container and the sterilization time still cannot be effectively controlled, whether effective sterilization is achieved or not still left unknown.

### SUMMARY

The disclosure is directed to a sterilization device with dose function used to generate an ozone dose matching the sterilization requirement and further control the ozone concentration and the sterilization time to effectively kill the germs and viruses.

According to one embodiment, a sterilization device with dose function is provided. The sterilization device includes a sterilization container, an ultraviolet sterilization module, an ozone sensing module, a sterilization time counter, a dose controller and an ozone removal module. The ultraviolet sterilization module is disposed in the sterilization container to sterilize an article by emitting an ultraviolet light. The ozone sensing module is used to sense an ozone concentration in the sterilization container. The sterilization time counter is used to calculate a sterilization time. The dose controller is used to receive a sensing signal of the ozone concentration and a counting signal of the sterilization time to obtain an ozone dose. The ozone removal module can purify the ozone passing by the article. During the sterilization time, the ozone removal module further prevents the ozone in the sterilization container from leaking to an external environment of the sterilization container. During the purification time, the ozone removal module works with the fan to reduce the ozone concentration in the sterilization container, such that the ozone in the sterilization container can match the regulatory standards of discharge (less than 60 ppb).

The above and other aspects of the disclosure will become better understood with regard to the following detailed description of the preferred but non-limiting embodiment(s). The following description is made with reference to the accompanying drawings.

### BRIEF DESCRIPTION OF THE DRAWINGS

FIG. 1 is a schematic diagram of a sterilization device with dose function according to an embodiment of the present disclosure;

FIG. 2 is a schematic diagram of a power supply circuit of a sterilization device with dose function according to an embodiment of the present disclosure;

FIG. 3 is a schematic diagram of a power supply circuit and a sensing circuit of a sterilization device with dose function according to an embodiment of the present disclosure; and

FIG. 4 is a schematic diagram of a control circuit of a sterilization device with dose function according to an embodiment of the present disclosure.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

Detailed descriptions of the disclosure are disclosed below with a number of embodiments. However, the disclosed embodiments are for explanatory and exemplary purposes only, not for limiting the scope of protection of the disclosure. Similar/identical designations are used to indicate similar/identical elements.

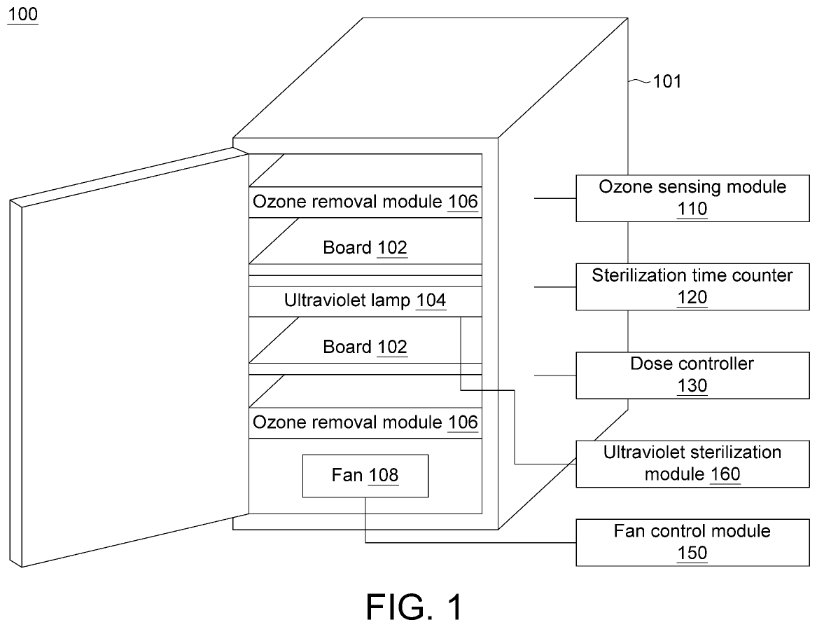

FIG. 1 is a schematic diagram of a sterilization device with dose function 100 according to an embodiment of the present disclosure is shown. In an embodiment, the sterilization device with dose function 100 is used to sterilize an article such as a face mask or an item of medical supplies. The article is disposed in a sterilization container 101, which has an ozone concentration matching the sterilization requirement. The sterilization container 101, such as a box having upper and lower boards 102, is used to receive a to-be-sterilized article. The ultraviolet lamp 104 is interposed between the upper and lower boards 102 and adjacent to the to-be-sterilized article. The ozone removal module 106 is disposed on the top side and the bottom side of the sterilization container 101. Besides, the fan 108 is disposed adjacent to the exit to dissipate the gas off the container, and further works with the ozone removal module 106 to remove the ozone.

In an embodiment, with a high ozone concentration, such as higher than or equivalent to 40 ppm, the article still can be quickly disinfected and sterilized within a short time, but the present embodiment is not limited thereto. Even when the ozone concentration is low, such as lower than 40 ppm, the article still can be effectively sterilized as long the sterilization time is long enough.

The sterilization device 100 of the present embodiment may further include an ozone sensing module 110, a sterilization time counter 120 and a dose controller 130. The ozone sensing module 110 is used to sense an ozone concentration. The sterilization time counter 120 is used to calculate a sterilization time. The dose controller 130 is used to receive a sensing signal of the ozone concentration and starts to calculate the sterilization time to obtain an ozone dose. Additionally, the sterilization device 100 of the present embodiment may further include an ozone removal module 106, which isolates the ozone from the external environment or works with the fan 108 to purify the ozone in the container. The ozone removal module 106, such as an oxygen-enriched carbonized material filter, can destroy and further remove ozone from the air, wherein the oxygen-enriched carbonized material may contain carbonyl-containing group, alkylol, and carbon having a sp2 hybrid orbital. The oxygen-enriched carbonized material can be formed of specific natural materials (such as rice husks, mushroom buns or water chestnuts) through carbonization, and can achieve an ozone removal rate as high as 99.9%.

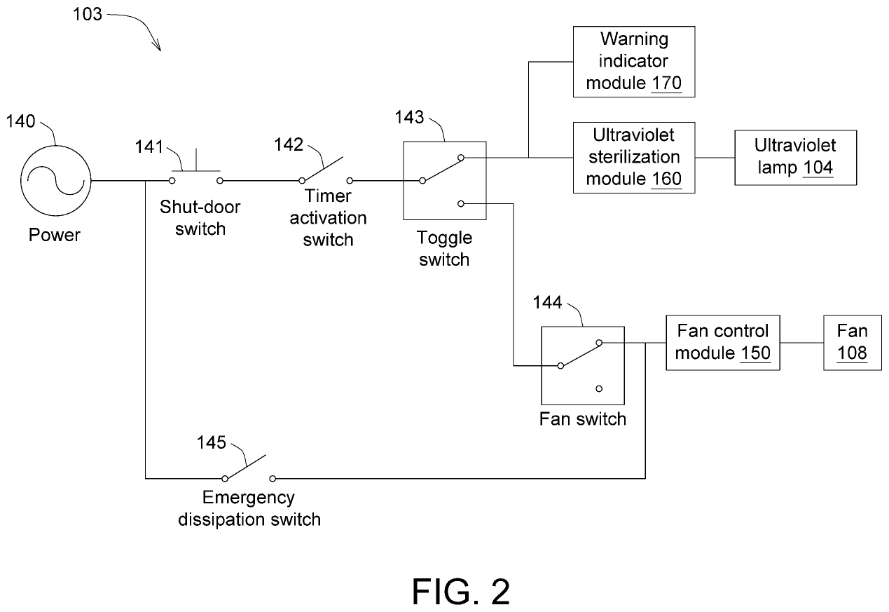

Referring to FIG. 2, a power supply circuit 103 of a sterilization device 100 with dose function according to an embodiment of the present disclosure is shown. The power supply circuit 103 is used to provide a power 140, such as an AC power or a DC power, to the sterilization device 100. The power supply circuit 103 may include a shut-door switch 141 connected to the power 140 to detect whether the door of the sterilization container 101 is closed. If the sterilization container 101 is not closed, the power 140 cannot be provided to the sterilization device 100. Thus, it can be assured that the ultraviolet lamp 104 will not be turned on and will not generate ozone, which may harm the user. Moreover, the power supply circuit 103 may include a timer activation switch 142 whose one end is connected to the shut-door switch 141. The timer activation switch 142 enables the dose controller 130 to start calculating the sterilization time. Also, the power supply circuit 103 may further include a toggle switch 143 whose one end is connected to the timer activation switch 142 or the shut-door switch 141 and the other end is connected to the ultraviolet sterilization module 160. When the dose controller 130 starts to calculate the sterilization time, the toggle switch 143 is turned on to electrify the ultraviolet sterilization module 160 and enable the ultraviolet lamp 104 to emit an ultraviolet light and generate photolysis reacted ozone. In an embodiment, the ultraviolet light within a wavelength range of 180 nm can effectively kill germs and viruses. As for the places not irradiated by the ultraviolet light, oxygen molecules in the air can be photolyzed using the ultraviolet light to generate a high concentration rate of ozone in the sterilization container. After sterilization, the used ozone can be removed by the ozone removal module 106 to avoid the ozone causing harm to the user or being leaked and becoming a secondary pollutant.

On the other hand, the power supply circuit 103 may further include a fan switch 144 whose one end is connected to the toggle switch 143 and the other end is connected to a fan control module 150. In an embodiment, after the ultraviolet sterilization module 160 completes sterilization, the ultraviolet lamp 104 is turned off, and the fan control module 150 activates the fan 108 to dissipate the ozone off the sterilization container and reduce the ozone concentration in the sterilization container 101. Generally speaking, if the ozone concentration at the exit can be lower than the regulatory standard (such as 60 ppb), the problem of secondary pollution, which occurs when the ozone generated by the ultraviolet light is directly dissipated without being fully reacted, will be resolved.

The power supply circuit 103 may further include an emergency dissipation switch 145 whose one end is connected to the power 140 and the other end is connected to the fan control module 150. When the power supply circuit 103 is powered off and the generated ozone is not fully removed, the emergency dissipation switch 145 can be turned on through human judgement, such that power can be provided to the fan control module 150 and the fan 108 can be activated to dissipate the ozone off the sterilization container 101 and reduce the ozone concentration in the sterilization container 101.

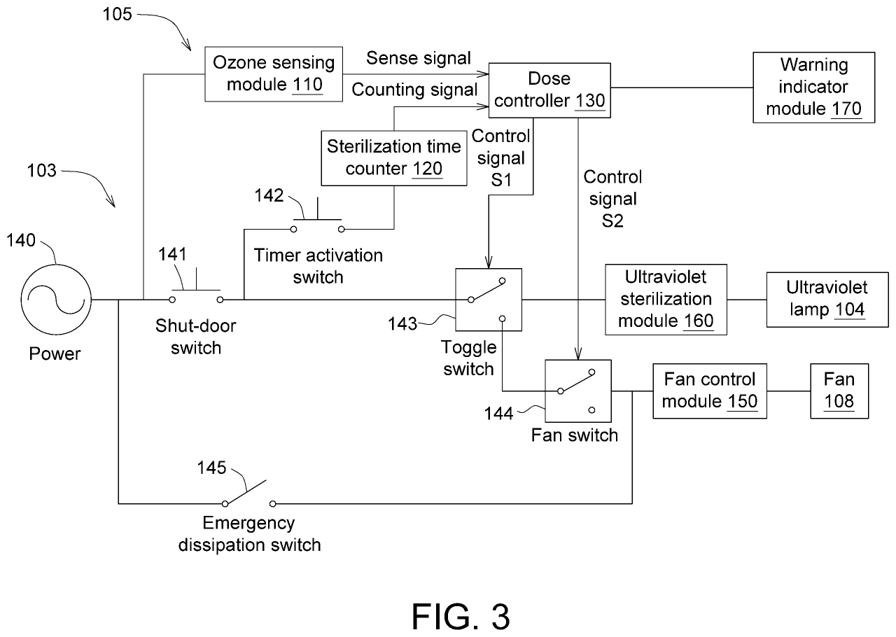

Referring to FIG. 3, a power supply circuit 103 and a sensing circuit 105 of a sterilization device 100 with dose function according to an embodiment of the present disclosure is shown. Descriptions of the power supply circuit 103 can be obtained with reference to FIG. 2 and are not repeated here. In an embodiment, the sensing circuit 105 includes an ozone sensing module 110, a sterilization time counter 120 and a dose controller 130. The ozone sensing module 110 is used to sense an ozone concentration and generate a sensing signal relative to the ozone concentration to the dose controller 130. One end of the sterilization time counter 120 is connected to the timer activation switch 142, and the other end is connected to the dose controller 130. When the timer activation switch 142 is turned on, the sterilization time counter 120 starts to calculate the sterilization time and generate a counting signal relative to the sterilization time to the dose controller 130.

The dose controller 130 is used to receive a sensing signal of the ozone concentration and a counting signal of the sterilization time to obtain an ozone dose. The ozone dose is a product of the ozone concentration multiplied by the sterilization time, which is continuously counted. When the ozone dose reaches a predetermined value and matches the sterilization requirement, such as longer than 47 min(mg/m³), the ozone dose can effectively kill germs and viruses. Since the required ozone dose varies with the species of viruses, the ozone concentration and the sterilization time can be adjusted according to the amount of the required ozone dose.

In an embodiment, given that the ozone concentration is equivalent to 40 ppm and the sterilization time is a quotient of the ozone dose 47 min(mg/m³) divided by the ozone concentration 40 ppm (about 78.3 mg/m³), the sterilization time is longer than 0.6 minutes. The above ozone concentration and sterilization time are for exemplary purpose only and can be adjusted according to the given conditions.

Refer to FIG. 3. One end of the toggle switch 143 is connected to the timer activation switch 142 or the shut-door switch 141 and the other end is connected to the ultraviolet sterilization module 160 to turn on/off the ultraviolet lamp 104. When the ozone dose reaches a predetermined value, the dose controller 130 emits a control signal S1 to a toggle switch 143 to turn off the ultraviolet lamp 104. Conversely, if the ozone dose does not reach the predetermined value, the ultraviolet lamp 104 remains in the turned-on state.

Refer to FIG. 3. When the ultraviolet lamp 104 is turned off and the sterilization time reaches a predetermined value, the dose controller 130 emits a control signal S2 to a fan switch 144 to activate the fan 108 to dissipate the ozone off the sterilization container and reduce the ozone concentration. Conversely, when the ozone concentration in the sterilization container 101 is lower than a safety setting value, such as lower than 6ppb, the dose controller 130 emits another control signal S2 to the fan switch 144 to turn off the fan 108.

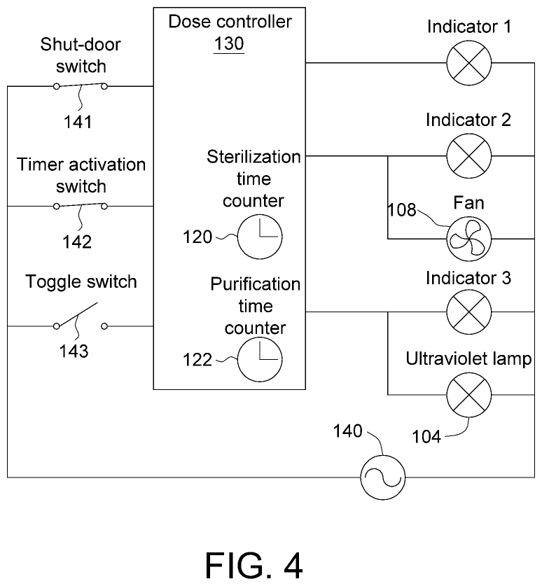

In an embodiment, the sensing circuit 105 may include a purification time counter 122 (referring to FIG. 4), which calculates the required time for the ozone concentration to be lower than a safety setting value after the sterilization process is completed. When the purification time reaches a predetermined value, the dose controller 130 emits a control signal S2 to the fan switch 144 to turn off the fan 108. The sterilization time counter 120 and the purification time counter 122 can be used together or separately. For example, firstly, the sterilization time counter 120 is activated to calculate the sterilization time, then the purification time counter 122 is activated to calculate the purification time. Since the sterilization time and the purification time do not overlap, the sterilization process and the purification process will not affect each other.

Referring to FIG. 4, a warning indicator module 170 of a sterilization device 100 with dose function according to an embodiment of the present disclosure is shown. In an embodiment, the warning indicator module 170 may include several indicators, such as power indicator (indicator 1), fan indicator (indicator 2) and ultraviolet light indicator (indicator 3). When the shut-door switch 141 is turned on, the power 140 is activated (indicator 1 is on), which implies that the door of the sterilization container 101 is closed and that the sterilization process and the purification process can be performed. Besides, when the timer activation switch 142 is turned on, the ultraviolet lamp 104 is activated (indicator 3 is on), which implies that the sterilization process is performed. Additionally, after the sterilization process is completed, the ultraviolet lamp 104 is turned off (indicator 3 is off), which implies that the sterilization process is completed, and the purification process can be performed subsequently. When the purification process just starts, the fan 108 is activated (indicator 2 is on) and will not be turned off (indicator 2 is off) until the purification process is completed. Moreover, when the power supply circuit 103 is powered off and the ozone concentration is not lower than a safety setting value, the fan 108 is activated (indicator 2 is on) to dissipate the ozone off the sterilization container and reduce the ozone concentration in the sterilization container 101.

According to the embodiments of the present disclosure, a sterilization device with dose function is provided. The ozone sensing module senses an ozone concentration and generates a sensing signal relative to the ozone concentration to the dose controller. The sterilization time counter calculates the sterilization time and generate a counting signal relative to the sterilization time to the dose controller to obtain an ozone dose matching the sterilization requirement. Thus, the sterilization device of the present disclosure can generate an ozone dose matching the sterilization requirement and can further control the ozone concentration and the sterilization time to effectively kill the germs and viruses. Moreover, the sterilization device of the present disclosure combines the sterilization process and the purification process to reduce the sterilization time, enhance the sterilization and purification effects and further avoid ozone becoming a secondary pollutant.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A sterilization device with dose function, comprising:
a sterilization container;
an ultraviolet sterilization module disposed in the sterilization container to sterilize an article by emitting an ultraviolet light;
an ozone sensing module used to sense an ozone concentration of the sterilization container;
a sterilization time counter used to calculate a sterilization time;
a dose controller used to receive a sensing signal of the ozone concentration and a counting signal of the sterilization time to obtain an ozone dose; and
an ozone removal module disposed in the sterilization container to remove or purify ozone to reduce the ozone concentration.

2. The sterilization device according to claim 1, wherein the ozone dose is longer than 47 min(mg/m³) to achieve sterilization effect.

3. The sterilization device according to claim 2, wherein the ozone concentration is higher than or equivalent to 40 ppm, and the sterilization time is longer than 0.6 minutes.

4. The sterilization device according to claim 1, wherein the article is adjacent to the ultraviolet sterilization module in the sterilization container, and the ozone removal module is disposed on a top side and a bottom side of the sterilization container.

5. The sterilization device according to claim 1, further comprising a shut-door switch whose one end is connected to a power and another end is connected to a timer activation switch, wherein the shut-door switch is used to detect whether the sterilization container is closed, and the timer activation switch enables the dose controller to start to calculate the sterilization time.

6. The sterilization device according to claim 5, further comprising a toggle switch whose one end is connected to the timer activation switch or the shut-door switch and another end of the toggle switch is connected to the ultraviolet sterilization module to turn on/off the ultraviolet lamp.

7. The sterilization device according to claim 6, wherein when the ozone dose reaches a predetermined value, the dose controller emits a control signal to the toggle switch to turn off the ultraviolet lamp.

8. The sterilization device according to claim 6, further comprising a fan switch whose one end is connected to the switch and another end of the fan switch is connected to a fan control module to turn on/off the fan.

9. The sterilization device according to claim 8, wherein when the ultraviolet lamp is turned off and the sterilization time reaches a predetermined value, the dose controller emits a control signal to the fan switch to turn on the fan to dissipate the ozone off the sterilization container and reduce the ozone concentration.

10. The sterilization device according to claim 8, further comprising an emergency dissipation switch whose one end is connected to the power, and another end of the emergency dissipation switch is connected to the fan control module.

11. The sterilization device according to claim 10, wherein when the power is powered off and the ozone concentration in the sterilization container is not lower than a safety setting value, the fan control module activates the fan to dissipate the ozone off the sterilization container and reduce the ozone concentration.

12. The sterilization device according to claim 11, further comprising a purification time counter used to calculate a required time for the ozone concentration to be lower than the safety setting value.
